(19) United States
(12) Patent Application Publication
(10) Pub. No.: US 2009/0171210 A1
(43) Pub. Date: Jul. 2, 2009

(54) SONOELECTRIC TOMOGRAPHY USING A FREQUENCY-SWEPT ULTRASONIC WAVE

(75) Inventor: Lihong Wang, Creve Coeur, MO (US)

Correspondence Address: PATRICK W. RASCHE (15060);ARMSTRONG TEASDALE, LLP, ONE METROPOLITAN SQUARE, SUITE 2600, SAINT LOUIS, MO, 63102-2740, US

(73) Assignee: WASHINGTON UNIVERSITY IN ST. LOUIS, St. Louis, MO (US)

(21) Appl. No.: 12/345,488

(22) Filed: Dec. 29, 2008

Related U.S. Application Data
(60) Provisional application No. 61/017,068, filed on Dec. 27, 2007.

(51) Int. Cl. A61B 8/13

(52) U.S. Cl. 600/443

(57) ABSTRACT

Sonoelectric tomography to achieve high-resolution bioelectric imaging. Bioelectrical signals originating from various locations in an object are ultrasonically encoded to carry different frequencies that vary with time via a frequency-swept signal or chirp. The frequency-sweeping parameters are chosen so that no frequencies are duplicated in the medium at any given time. The frequency distribution is decoded, for example, by means of the Fourier transformation to recover a bioelectric image with high spatial resolution. The spatial resolution of the image is defined by the ultrasonic frequency parameters, whereas the image contrast is derived from the bioelectric signals. In an embodiment, an ultrasonic transducer transmits a frequency-swept (e.g., chirped) ultrasonic wave into the region of interest in the tissue. The ultrasonic wave may be focused to achieve high transverse resolution within the focal zone.

### CROSS REFERENCE TO RELATED APPLICATION

This application claims the benefit of U.S. Provisional Application No. 61/017,018 filed Dec. 27, 2007, which is hereby incorporated by reference in its entirety.

### BACKGROUND

High-resolution imaging of bioelectric signals arising from physiological (such as brain or cardiac) activity is lacking in the art. Currently, no non-invasive imaging modality can image intrinsic bioelectric sources in living tissues with high spatial resolution. While electroencephalography (EEG) and electrocardiography (ECG) can image endogenous bioelectric signals with high temporal resolution, spatial resolution is poor. An advancement in the domain of bioelectric imaging is therefore critical in overcoming the limitations of the prior art.

### BRIEF DESCRIPTION OF THE DRAWINGS

FIG. 1(a) is a block diagram showing a scanning sonoelectric tomography apparatus with one-dimensional encoding.

FIG. 1(b) is a graphical representation of the instantaneous ultrasonic frequencies versus the ultrasonic axis.

FIG. 2 is a block diagram illustrating conversion of a time-variant frequency into a time-invariant frequency with a mixer.

FIG. 3 is a diagram showing a scanning sonoelectric tomography apparatus with two-dimensional or three-dimensional encoding

FIG. 4 is a flow chart illustrating operations performed in an embodiment of the invention.

Corresponding reference characters indicate corresponding parts throughout the drawings.

### DETAILED DESCRIPTION

Embodiments of the invention provide sonoelectric tomography (SET) to achieve high-resolution bioelectric imaging. In an embodiment, intrinsic bioelectric signals originating from various tissue locations are ultrasonically encoded (e.g., modulated or tagged) with different frequencies. When the bioelectric signals are modeled as current dipoles, the ultrasonic wave vibrates the dipoles at the local instantaneous ultrasonic frequencies. From the time-domain electric signals recorded outside the tissue, such a frequency distribution is decoded, for example, by means of the Fourier transformation to recover a bioelectric image of the tissue with high spatial resolution. The spatial resolution of the image is defined by the ultrasonic parameters, whereas the contrast is derived from the bioelectricity. Also, the frequency-sweeping parameters are chosen so that no frequencies are duplicated in the medium at any given time.

Ultrasonically encoding the bioelectric sources allows bioelectric imaging with high spatial and temporal resolution. For example, aspects of the invention allow direct volumetric imaging of neural or cardiac activity with spatial resolution (in millimeters) equivalent to that of magnetic resonance imaging and temporal resolution (in milliseconds) equivalent to that of electroencephalography.

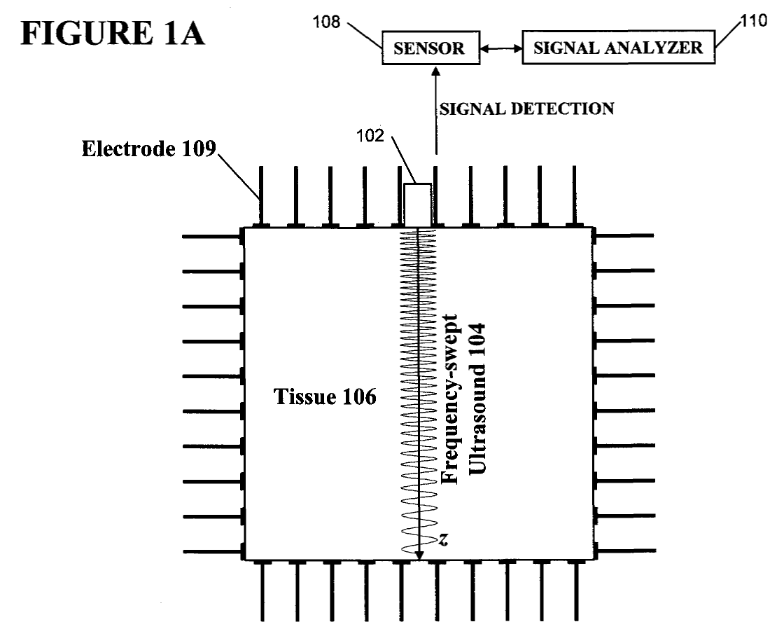

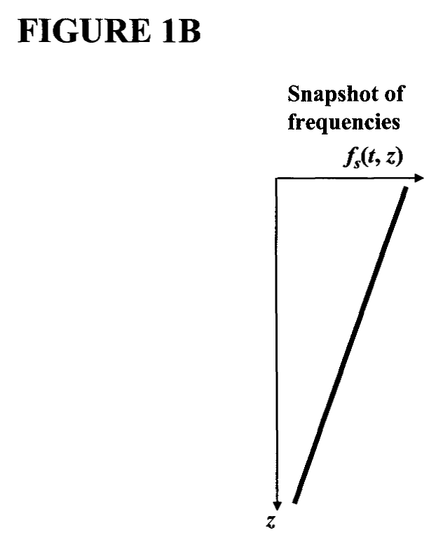

The ultrasonic wave encodes the bioelectric signals along the ultrasonic axis with various frequencies. In an embodiment, an ultrasonic transducer 102 transmits a frequency-swept (e.g., chirped) ultrasonic wave 104 or signal into the region of interest in the tissue 106 (see FIG. 1A). The frequency of the ultrasonic wave 104 at the ultrasonic transducer 102 varies linearly with time. Further, the ultrasonic wave 104 may be focused to achieve high transverse resolution within the focal zone.

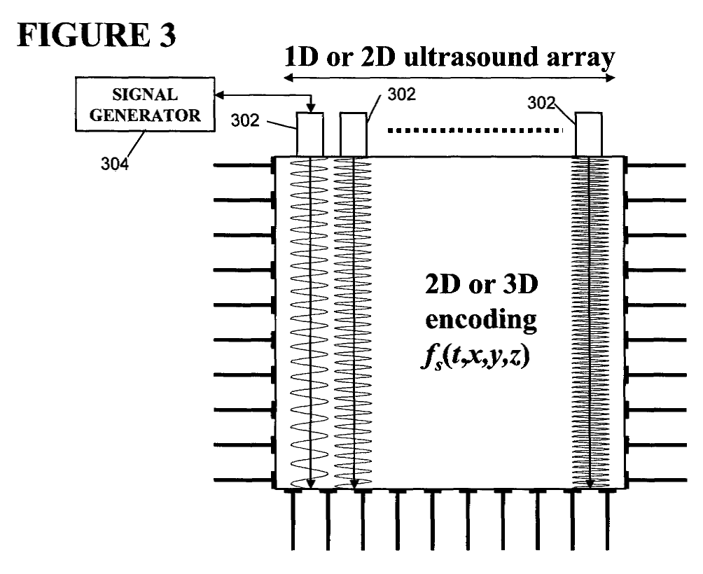

Referring again to FIG. 1A, the ultrasonic wave 104 is transmitted by the transducer 102 by means of a signal generator (see signal generator in FIG. 3) in an object of interest such as body tissue 106. The ultrasonic wave 104 is a frequency-swept signal over a finite time period. As the ultrasonic wave 104 penetrates through the tissue 106, the frequency varying ultrasonic wave 104 encounters bioelectric signals that are generated by the body tissue 106. As a consequence of the interaction between the ultrasonic wave 104 and the bioelectric signals, the bioelectric signals are encoded with the ultrasonic wave 104 to generate modulated signals of varying frequencies. Because of the finite speed of sound (vₛ≈1.5 mm/μs in soft tissue), the instantaneous frequencies of the ultrasonic wave 104 form a gradient along the ultrasonic axis (see FIG. 1B). Because bioelectric signals travel at the speed of light, the ultrasound-modulated bioelectric signals (e.g., encoded or tagged) reflect the local instantaneous ultrasonic frequencies.

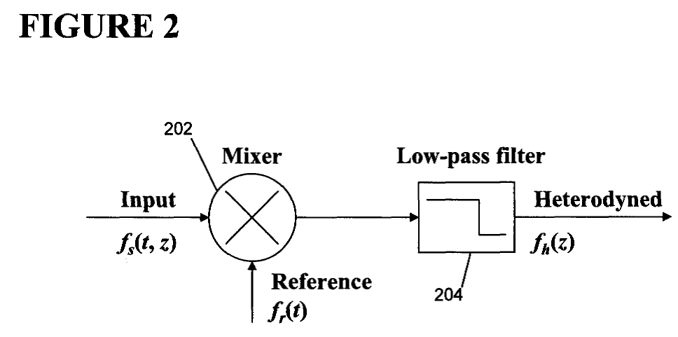

The encoded bioelectric signals are radiated from the tissue 106 and detected by a sensor 108 connected to one or more of a plurality of electrodes 109. The sensor 108 or other electronic detection system is similar to the systems used in EEG and ECG. The electric signals received from all electrodes 109 are amplified and filtered by means of individual electronic machinery known in the art. The encoded electric signal originating from each ultrasonic axial position has a time-variant frequency, which is converted (e.g., heterodyned) into a time-invariant frequency by means of a signal analyzer 110 in FIG. 1A or a mixer 202 followed by a low-pass filter such as shown in FIG. 2. An embodiment includes a low-pass filter 204 to eliminate high frequencies. The frequencies of the signals are marked accordingly in FIG. 2. The conversion is shown mathematically below. The ultrasound-encoded bioelectric signals along the ultrasonic axis (z) have an instantaneous frequency as in equation (1) below.

fₛ(t,z)=aₛ+b(t−(z−z₀)/vₛ) for t≧(z−z₀)/vₛ, (1)

where aₛ denotes the starting frequency, b the sweep rate, t time, and z₀ is a reference point. The reference chirp signal has an instantaneous frequency fᵣ(t)=aᵣ+bt. Therefore, the heterodyned modulated signal has an instantaneous frequency as shown in equation (2) below.

fₕ(z)=|fₛ(t,z)−fᵣ(t)|=|aₛ−aᵣ−b(z−z₀)/vₛ|, (2)

which is independent of time (t). The absolute value operator can accommodate positive or negative values depending on the chosen parameters.

The one-to-one correspondence between the heterodyned frequency and depth along the ultrasonic axis (e.g., the z coordinate) enables imaging. For example, the time-domain heterodyned signal is acquired by a computing device or signal analyzer 110 and Fourier transformed into a spectrum. The frequency in the spectrum corresponding to fₕ is converted into coordinate z using equation (2) above. This converts the frequency spectrum into a position spectrum. The amplitude of each position spectrum is a one-dimensional (1D) image of bioelectric signals along z. Therefore, a 1D image along the ultrasonic axis is formed. The axial resolution z_{R} is determined by the chirp bandwidth Δfₕ in equation (3) below.

z_{R}=vₛ/Δfₕ, (3)

whereas the transverse resolution is provided by the beam diameter. The ultrasonic wave 104 may be focused to achieve high transverse resolution within the focal zone. Because the spatial resolution is determined solely by ultrasonic parameters such as chirp bandwidth and beam width, the electrodes 109 may have relatively large contact areas and the amplitudes of the position spectra from all electrodes 109 may be averaged for signal enhancement such as improving the signal to noise ratio (SNR). At least because electric signals of different frequencies do not cancel, frequency encoding of bioelectric signals avoids the current-dipole signal cancellation encountered in EEG.

A 2D image is obtained by scanning the ultrasonic transducer 102 along a line. At each stop along the line, a 1D image is acquired. All the 1D images are pieced together to form a 2D image. Similarly, a 3D image is acquired by raster scanning the ultrasonic transducer 102. In an alternate embodiment, ultrasonic pulses may be used in the place of a continuous signal. Consequently, the axial image resolution is a function of the pulse duration.

Referring next to FIG. 3, a 2D cross-section or a 3D volume is imaged simultaneously. The single-element ultrasonic transducer 102 in FIG. 1A is replaced by a 1D or 2D array of ultrasonic transducers 302. Each transducer 302 is driven by a chirp signal (e.g., from a function generator or signal generator 304) that spans a different frequency range so that each instantaneous frequency in the tissue is unique at any point in time. In an embodiment, the rest of the system remains the same as illustrated and described with reference to FIG. 1A.

Embodiments of the invention are expected to provide millimeter spatial resolution, which is scalable with targeted imaging depth. The spatial resolution of the image is comparable with the acoustic wavelength. For example, if a 1-MHz ultrasonic frequency range is used, the spatial resolution should be ˜1.5 mm. However, the spatial resolution may be scaled according to the applications in mind. The spatial resolution may be enhanced at the expense of tissue penetration by increasing the ultrasonic bandwidth, and vice versa. For example, if the ultrasonic frequency is increased to 10 or 100 MHz, the expected resolution would be on the order of 150 or 15 μm respectively. In other words, sonoelectric tomography provides multiscale imaging of, for example, various levels of brain function.

Aspects of the invention provide millisecond temporal resolution. According to the duration of bioelectric signals (e.g., in milliseconds), the speed of sound (1.5 mm/μs), and the frequency of the encoding ultrasound (e.g., in megahertz), the data acquisition time is expected to be on the millisecond scale. The millisecond bioelectric variation gives the ultrasound enough time to modulate the bioelectric signal. The 1.5 m/ms speed of sound allows ultrasound to occupy the region of interest rapidly within the millisecond window of bioelectric variation.

Bedside imaging is feasible with aspects of the invention. As both ultrasound imaging and EEG/ECG systems are much more compact than CT/MRI, embodiments of the invention may be constructed relatively compactly.

Embodiments of the invention enable 3D direct imaging of bioelectricity without the use of either downstream secondary surrogates or exogenous contrast agents. Ultrasound encoding of current dipole sources provides ultrasound-limited spatial resolution (e.g., in millimeters) and real-time data acquisition (e.g., in milliseconds). Because electric signals of different frequencies do not cancel, frequency encoding of bioelectric signals avoids signal cancellation as encountered in EEG/MEG.

The applications of sonoelectric tomography range in multiple aspects of medical examination including but not limited to diagnosing and staging epilepsy (e.g., enhancing preoperative staging of epileptics by accurately localizing seizure foci to maximize excisions and minimize collateral damage) and other brain abnormalities, imaging cardiac diseases (e.g., overlaying images from sonoelectric tomography and cardiac ultrasonography), imaging the fetus transabdominally, understanding cognitive neurosciences, or enabling scientists and clinicians to meet challenges related to the brain and the heart as well as other vital organs.

Alternatively or in addition, the mechanical scanning is replaced with electronic scanning using known linear or 2D ultrasonic arrays. Further, when the brain is imaged using embodiments of the invention, correction of wavefront aberration due to the skull may be implemented using known ultrasound array technologies.

Alternatively or in addition, single-frequency continuous-wave ultrasonic signals instead of chirped signals are used to encode bioelectric signals. In such embodiments, because the chirp bandwidth Δfₕ reduces to zero, the axial resolution z_{R} approaches infinity.

In embodiments of the invention, ultrasonic radiation force provides micron scale acoustic displacement due to momentum transfer. Such displacement may be used to encode bioelectric signals as well. In such embodiments, an ultrasonic radiation force is generated. The generated force varies in frequency over time. The generated force is applied to an object (e.g., a body) along an axis to ultrasonically modulate bioelectric signals at a plurality of locations within the object along the axis. Each of the bioelectric signals is modulated with one of the frequencies in the generated force. Time-domain electric signals radiating from the object are received (e.g., via a sensor placed external to the object). The received time-domain electric signals correspond to the ultrasonically modulated bioelectric signals. The received time-domain electric signals are converted into a time-invariant frequency distribution (e.g., via a low-pass filter) to identify portions of the object along the axis. A bioelectric image is generated with high-spatial resolution from the identified portions of the object.

The figures illustrated and described herein along with the equations and examples constitute exemplary means for generating an ultrasonic frequency-swept signal, exemplary means for detecting a time-variant electric signal radiating from a body or other object, and exemplary means for generating a bioelectric image from time-variant electric signals radiating from the body or other object. However, equivalent means not specifically described herein are contemplated in embodiments of the invention. For example, exemplary means for generating the ultrasonic frequency-swept signal include a function generator. Further, embodiments of the invention are operable with means known in the art other than a Fourier transform for converting time-domain signals into frequency-domain signals. In another example, a computing device constitutes the exemplary means for generating the bioelectric image.

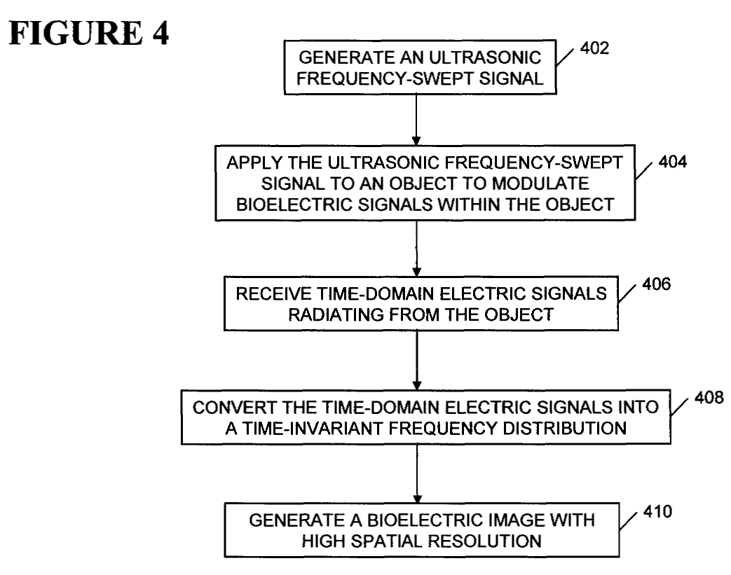

Referring next to FIG. 4, a flow chart illustrated operations performed in embodiments of the invention. The operations illustrated may be performed by one or more devices separately, or performed or controlled by a computing device. At 402, an ultrasonic frequency-swept signal is generated. At 404, the ultrasonic frequency-swept signal is applied to an object to modulate bioelectric signals within the object. At 406, time-domain electric signals radiating from the object are detected, recorded, or otherwise received. At 408, the received time-domain electric signals are converted into a time-invariant frequency distribution. At 410, a bioelectric image with high spatial resolution is generated from the time-invariant frequency distribution.

### Exemplary Operating Environment

A computing device or computer such as described herein has one or more processors or processing units and a system memory. The computer typically has at least some form of computer readable media. Computer readable media, which include both volatile and nonvolatile media, removable and non-removable media, may be any available medium that may be accessed by computer. By way of example and not limitation, computer readable media comprise computer storage media and communication media. Computer storage media include volatile and nonvolatile, removable and non-removable media implemented in any method or technology for storage of information such as computer readable instructions, data structures, program modules or other data. For example, computer storage media include RAM, ROM, EEPROM, flash memory or other memory technology, CD-ROM, digital versatile disks (DVD) or other optical disk storage, magnetic cassettes, magnetic tape, magnetic disk storage or other magnetic storage devices, or any other medium that may be used to store the desired information and that may be accessed by computer. Communication media typically embody computer readable instructions, data structures, program modules, or other data in a modulated data signal such as a carrier wave or other transport mechanism and include any information delivery media. Those skilled in the art are familiar with the modulated data signal, which has one or more of its characteristics set or changed in such a manner as to encode information in the signal. Wired media, such as a wired network or direct-wired connection, and wireless media, such as acoustic, RF, infrared, and other wireless media, are examples of communication media. Combinations of any of the above are also included within the scope of computer readable media.

Although described in connection with an exemplary computing system environment, embodiments of the invention are operational with numerous other general purpose or special purpose computing system environments or configurations.

Embodiments of the invention may be described in the general context of computer-executable instructions, such as program modules, executed by one or more computers or other devices. The computer-executable instructions may be organized into one or more computer-executable components or modules. Generally, program modules include, but are not limited to, routines, programs, objects, components, and data structures that perform particular tasks or implement particular abstract data types. Aspects of the invention may be implemented with any number and organization of such components or modules. For example, aspects of the invention are not limited to the specific computer-executable instructions or the specific components or modules illustrated in the figures and described herein. Other embodiments of the invention may include different computer-executable instructions or components having more or less functionality than illustrated and described herein. Aspects of the invention may also be practiced in distributed computing environments where tasks are performed by remote processing devices that are linked through a communications network. In a distributed computing environment, program modules may be located in both local and remote computer storage media including memory storage devices.

The order of execution or performance of the operations in embodiments of the invention illustrated and described herein is not essential, unless otherwise specified. That is, the operations may be performed in any order, unless otherwise specified, and embodiments of the invention may include additional or fewer operations than those disclosed herein. For example, it is contemplated that executing or performing a particular operation before, contemporaneously with, or after another operation is within the scope of aspects of the invention.

When introducing elements of aspects of the invention or the embodiments thereof, the articles “a,” “an,” “the,” and “said” are intended to mean that there are one or more of the elements. The terms “comprising,” “including,” and “having” are intended to be inclusive and mean that there may be additional elements other than the listed elements.

Having described aspects of the invention in detail, it will be apparent that modifications and variations are possible without departing from the scope of aspects of the invention as defined in the appended claims. As various changes could be made in the above constructions, products, and methods without departing from the scope of aspects of the invention, it is intended that all matter contained in the above description and shown in the accompanying drawings shall be interpreted as illustrative and not in a limiting sense.

What is claimed is:

1. A system comprising:
an ultrasonic transducer configured to generate an ultrasonic frequency-swept signal, said generated ultrasonic frequency-swept signal varying in frequency over time, wherein the generated ultrasonic frequency-swept signal penetrates an object to ultrasonically modulate bioelectric signals radiating from the object, each of said bioelectric signals being modulated with one of the varying frequencies, said bioelectric signals originating from a plurality of locations within the object;
a sensor configured to detect time-domain electric signals radiating from the object, said detected time-domain electric signals corresponding to the ultrasonically modulated bioelectric signals; and
an analyzer coupled to said sensor and configured to decode a frequency distribution from the detected time-domain electric signals, said signal analyzer recovering, from the decoded frequency distribution, a bioelectric image of a portion of the object with high spatial resolution.

2. The system of claim 1, wherein the bioelectric signals comprise current dipoles, and wherein the generated ultrasonic frequency-swept signal vibrates each of the dipoles at the frequency corresponding to a location of the dipole in the object.

3. The system of claim 1, wherein the spatial resolution of the bioelectric image is defined by the ultrasonic parameters.

4. The system of claim 1, wherein a contrast in the bioelectric image is derived from the bioelectricity of the object.

5. The system of claim 1, wherein the detected time-domain electric signals comprise time-variant frequencies, and wherein the signal analyzer converts the time-variant frequencies into a time-invariant frequencies.

6. The system of claim 1, wherein the signal analyzer performs a Fourier transformation to decode the frequency distribution.

7. The system of claim 1, wherein the bioelectric signal is generated by the object.

8. The system of claim 1, wherein the ultrasonic transducer comprises a plurality of ultrasonic transducers each configured to generate an ultrasonic frequency-swept signal along an axis to produce a two-dimensional bioelectric image.

9. The system of claim 1, further comprising means for generating the ultrasonic frequency-swept signal

10. The system of claim 1, further comprising means for detecting the time-variant electric signals radiating from the body.

11. The system of claim 1, further comprising means for generating the bioelectric image from the detected time-variant electric signals.

12. A method for ultrasound frequency-encoded electric tomography, said method comprising:
generating an ultrasonic frequency-swept signal, said generated signal varying in frequency over time;
applying the generated ultrasonic frequency-swept signal to an object along an axis to ultrasonically modulate bioelectric signals at a plurality of locations within the object along the axis, each of said bioelectric signals being modulated with one of the frequencies in the generated ultrasonic frequency-swept signal;
receiving time-domain electric signals radiating from the object, said received time-domain electric signals corresponding to the ultrasonically modulated bioelectric signals;
converting the received time-domain electric signals into a time-invariant frequency distribution to identify portions of the object along the axis; and
generating a bioelectric image with high-spatial resolution from the identified portions of the object.

13. The method of claim 12, wherein the object comprises body tissue, and wherein positive and negative charges within the bioelectric signals are separated biologically.

14. The method of claim 12, wherein receiving the time-domain electric signals comprises measuring a voltage from the object.

15. The method of claim 12, wherein the frequencies within the generated ultrasonic frequency-swept signal are greater than frequencies of the intrinsic bioelectric signals prior to modulation.

16. The method of claim 12, wherein the generated ultrasonic frequency-swept signal varies linearly over time.

17. A method for electric tomography using ultrasonic radiation force, said method comprising:
generating an ultrasonic radiation force, said generated force varying in frequency over time;
applying the generated force to an object along an axis to ultrasonically modulate bioelectric signals at a plurality of locations within the object along the axis, each of said bioelectric signals being modulated with one of the frequencies in the generated force;
receiving time-domain electric signals radiating from the object, said received time-domain electric signals corresponding to the ultrasonically modulated bioelectric signals;
converting the received time-domain electric signals into a time-invariant frequency distribution to identify portions of the object along the axis; and
generating a bioelectric image with high-spatial resolution from the identified portions of the object.

18. The method of claim 17, wherein applying the generated force to the object comprises applying the generated force to provide acoustic displacement due to momentum transfer.

19. The method of claim 17, wherein converting the received time-domain electric signals comprises heterodyning the received time-domain electric signals via a low-pass filter.

20. The method of claim 17, wherein receiving the time-domain electric signals comprising receiving the time-domain electric signals via a sensor placed external to the object.
